(19) United States
(12) Patent Application Publication
(10) Pub. No.: US 2003/0229279 A1
(43) Pub. Date: Dec. 11, 2003

(54) Determination of deformations of surgical tools

(76) Inventors: Christoph Amstutz, St. Moritz (CH); Lutz-Peter Nolte, Thun (CH)

Correspondence Address: PENNIE & EDMONDS LLP, 1667 K STREET NW, SUITE 1000, WASHINGTON, DC, 20006

(21) Appl. No.: 10/425,949

(22) Filed: Apr. 30, 2003

Related U.S. Application Data
(63) Continuation of application No. PCT/CH00/00587, filed on Nov. 3, 2000.

(51) Int. Cl. A61B 5/05

(52) U.S. Cl. 600/424

(57) ABSTRACT

The present invention relates to a method of determining deformations of instruments having a tool with a central axis and a tool holder comprises the steps of establishing a mathematical relationship between the tool holder and the body, displacing the tool relative to the body, determining the relative position and spatial orientation of the tool with respect to the body, establishing mechanical contact between the tool and the body and/or entering the body with the tool and determining the deformation of the tool with respect to the virtual, non-deformed tool resulting from the load application onto the tool.

The measurements in order to determine the deformation of the tool may be performed on the one hand by measuring the position and orientation of the tool holder and the body with respect to an on-site three-dimensional system of coordinates or on the other hand by means of forces gauges apt to measure the loads being effective on the tool holder.

### RELATED APPLICATIONS

The present application is a continuation of international application no. PCT/CH00/00587, filed Nov. 3, 2000, and incorporated entirely herein by reference.

### FIELD OF THE INVENTION

The present invention relates to a method and device for determining deformations of surgical tools.

### BACKGROUND

Computer assisted surgery systems (CAS systems) that are provided with a computer and a position measurement device in order to measure the position of surgical instruments or devices, which are displaceable within the operation area, are disclosed e.g. in U.S. Pat. No. 5,682,886 to Delp. CAS systems may have a memory in order to store medical images such as e.g. X-rays, computertomographs or MR images (Magnetic Resonance images). The medical images may be gathered preoperatively or intraoperatively.

In computer assisted orthopedic surgery systems, tracked components such as a surgical instrument or stereotactic tools usually are assumed to be accurately represented by a rigid body. However, during surgical action, some of these components may undergo considerable deformation. The deformation leads to a position difference between a real position and an undeformed position of the tool. This may be relevant for any slender linear tool. Drills are of particular concern because of the slender drill bit geometry, the relatively high applied forces, and the serious potential hazards. In conclusion, the accuracy of CAS systems for determining the position of tools such as drill drives may be limited.

### SUMMARY OF THE INVENTION

One aspect of the present invention relates to a method for determining deformation of a surgical tool. In one embodiment, the method of the invention may determine axial deformations, such as those caused by application of a non-axial load onto the surgical tool or a tool holder holding the surgical tool during an operation. For example, during a drilling process, a load may be applied to the drill drive by the operators hand, the load being composed of force and moment. Through the drill drive (tool holder) and drill bit (tool), this load is transmitted to the drilled target object (the body). The drill drive and target object are considered to be rigid structures, whereas the elasticity of the drill bit has to be taken into account.

Another embodiment of the invention relates to a method of determining a deformation of an instrument comprising (i) a tool holder and (ii) a tool comprising a central axis, the tool having an un-deformed state. The method may comprise (a) determining a position, in three-dimensional space, of a first point of a body to be operated on using the tool. Determining the position of the first point of the body may comprise detecting energy radiated by each of at least three energy emitters. The energy may pass between the at least three energy emitters and a detector, which may be mechanically separate from the at least three energy emitters. The method may comprise determining a position, in three-dimensional space, of a proximal portion of the tool, wherein the position of the proximal portion of the tool is determined based upon the position of the first point of the body. For example, the proximal portion of the tool may be a point along the central axis of the tool.

The method may comprise determining a position, in three-dimensional space, of a distal portion of the tool, which determining step may comprise detecting energy radiated by each of at least three energy emitters, the energy passing between the at least three energy emitters and a detector, the three energy emitters and the detector being mechanically separate from one another. The at least three energy emitters used in determining the position of the distal portion of the tool may be the same as or different from energy emitters used in determining the position of the first point of the body to be operated on using the tool.

The method preferably comprises determining a difference between (i) a relative position of the proximal portion of the tool with respect to the distal portion of the tool as determined from steps (b) and (c) and (ii) a relative position of the proximal portion of the tool with respect to the distal portion of the tool when the tool is in the un-deformed state.

In yet another embodiment, the method comprises the steps of:

A) establishing a mathematical relationship between a tool holder with a tool and a body wherein the geometry and the material properties of the tool are known. The mathematical relationship preferably consists of a measured position and spatial orientation each of the tool holder and the body with respect to a three-dimensional system of coordinates. The measurement of the position and spatial orientation of the tool holder and the body may be performed with a position measurement device and the mathematical relationship may be computed in the form of determining coordinates of defined points within the three-dimensional system of coordinates by means of a computer connected to the position measurement device;

B) displacing the tool relative to the body;

C) determining the relative position and spatial orientation of the tool with respect to the body. This step may be performed by means of measuring the position and spatial orientation of the body and the tool holder having a known relative position to the tool within the three-dimensional systems of coordinates;

D) establishing mechanical contact between the tool and the body and/or entering the body with the tool, e.g. during a drilling process; and

E) determining the deformation of the tool with respect to a virtual non-deformed tool resulting from the load application onto the tool.

In a preferred embodiment of the method according to the invention the measurements in order to determine the deformation of the tool are performed by measuring the position and orientation of the tool holder and the body with respect to an on-site three-dimensional system of coordinates.

In another embodiment of the method according to the invention the measurements in order to determine the deformation of the tool are performed by means of force gauges apt to measure the loads being effective on the tool holder.

Another embodiment of the invention relates to a method of determining deformations of preferably surgical instruments comprising a tool holder and a tool with a central axis. The method comprises the steps of establishing a mathematical relationship between the tool holder and a body to be operated on using the tool, wherein the geometry and the material properties of the tool are known. The tool is displaced relative to the body. The relative position and spatial orientation of the tool with respect to the body is determined. Mechanical contact may be established between the tool and the body. Alternatively, or in addition, the body may be entered with the tool.

Preferably, the method comprises the step of determining the deformation of the tool resulting from the load application to the tool with respect to the virtual, non-deformed tool.

In one embodiment, the establishment of the mathematical relationship between the tool holder and the body comprises measuring the position and spatial orientation of a first reference base attached to the tool holder and of a second reference base attached to the body, wherein position and orientation of the reference bases are measured with respect to an on-site three-dimensional system of coordinates.

The deformation of the tool may be a result of a load application perpendicular to the central axis of the tool. The deformation may be determined with respect to a rigid body trajectory of a virtual undetected tool. The central axis of the deflected tool and the rigid body trajectory may be represented at a display unit of a computer.

The measurements in order to determine the deformation of the tool may be performed by measuring the position and orientation of the tool holder and the body with respect to an on-site three-dimensional system of coordinates (6).

The measurements in order to determine the deformation of the tool may be performed using force gauges configured to measure the loads being effective on the tool holder.

Another embodiment of the invention relates to a device for determining deformations of surgical instruments due to loads applied to the surgical instruments. In one embodiment, the device comprises (a) a tool, which is preferably a surgical tool with a central axis, (b) a tool holder, wherein the tool is connectable to the tool holder at one end of the tool, (c) a position measurement device configured to determine the on-site position of the tool holder and a body to be treated, and (d) a computer or computing means configured to establish a mathematical relationship between the position and spatial orientation of the tool holder and the position and spatial orientation of the body.

The computer or computing means may comprise software configured to determine the deformation of the tool when treating the body with the tool. The computer or computing means may comprise software configured to represent the undeformed tool and/or the deformed tool at a display unit.

The device may comprise a measuring device configured to emit signals referenced to forces and moments applied to a body through the tool.

The computer or computing means may comprise software configured to determine the deformation of the tool through application of a kinematic model in order to determine an interdependence of forces applied onto the tool and the deformation of the tool.

The tool holder may comprise force gauges configured to perform the measurements of loads being effective on the tool holder when treating the body. The force gauges may comprise load cells. The force gauges may comprise wire strain gauges.

In another embodiment of a device of the invention, the device comprises a tool, particularly a surgical tool, a tool holder connectable to the tool at one end of the tool, a position measurement device in order to determine the on-site position of the tool holder and a body to be treated and computing means in order to establish the mathematical relationship between the position and spatial orientation of the tool holder and the position and spatial orientation of the body. The computing means particularly comprise software apt to determine the deformation of the tool when treating the body with the tool.

In another embodiment of the device according to the invention the tool holder comprises force gauges in order to perform the measurements of loads being effective on the tool holder when treating the body, whereby the force gauges may comprise load cells or wire strain gauges.

The advantages achieved by the invention are essentially to be seen in the fact that, thanks to the method and the device according to the invention it is possible to incorporate and consider deformations of the surgical instrument, particularly axial deflections effected through the application of a non-axial load onto the tool holder during the surgical operation.

### BRIEF DESCRIPTION OF THE DRAWINGS

The present invention is discussed below in reference to the drawings, in which:

FIG. 1 shows a first embodiment of a device comprising a surgical drill configured to drill a hole into a bone in accordance with the present invention;

FIGS. 2-6 show a schematic representation of a method in accordance with the present invention;

FIG. 7 shows a schematic representation of a surgical instrument configured to determine a deformation of the instrument, the surgical instrument configured to optically determine a position of the instrument in accordance with the present invention; and

FIG. 8 shows a schematic representation of a surgical instrument configured to determine a deformation of the instrument, the surgical instrument configured to determine a force acting upon the surgical instrument in accordance with the present invention.

### DESCRIPTION OF THE PREFERRED EMBODIMENTS

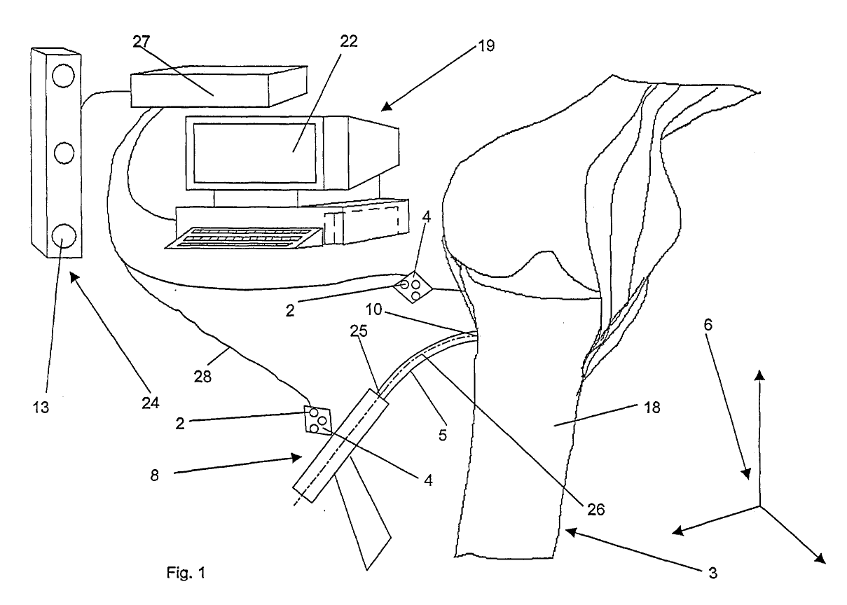

FIG. 1 shows an embodiment of the device according to the invention comprising a drilling gear as tool holder 8, a surgical drill as tool 5 having a central axis 26 and being clamped within the drilling gear at its fixed or distal end 25. The tool 5 is applied to drill a hole into a body 3 which may be a tibia 18. Both, the tibia 18 and the tool holder 8 are provided with dynamic reference bases 1,4. The tool 5 enters the tibia 18 at an entry point 10 and may deflect during the drilling process thereby deforming, such as by bending, the tool from an undeformed state of the tool. Preferred methods and devices in accordance with the present invention relate to determination of the deformation of the tool.

During a drilling process the hand of the drill operator may apply a force F at a distance d from the drill axis. Each component including the drill bit has to be in equilibrium with respect to force and moment. The Euler-Bernoulli theory of slender beams provides an accurate model for the drill bit bending.

In case of deflection of e.g. the drill bit relative to the axis of the drill drive the above determination procedure may be effected via a softwaretool named FlexDrill implemented on a computer.

As a position measurement device 24 an optoelectronic device may be used. Dynamic reference bases 1; 4 are provided with markers 2 that may be IRED's and the position of which is detected through the three cameras 13 of the position measurement device 24. The position and orientation of the tool holder 8 and the entry point 10 at the tibia 18 within the on-site three-dimensional system of coordinates 6 is determined by means of measuring the position of each of the markers 2 attached at both dynamic reference bases 1; 4 with respect to a three-dimensional system of coordinates which may be the on-site three-dimensional system of coordinates 6 and calculating the position of the entry point 10 at the tibia 18 and the position and orientation of the tool holder 8 by means of the computer 19 connected to the position measurement device 24. In order to transmit the relevant data the computer 19, the position measurement device 24, the system control unit 27 and the dynamic reference bases 1; 4 are connected by cables 28.

Both the drill drive and the target body may be equipped with reference bases that are provided with markers e.g. infrared light emitting diodes (IREDs), which are tracked by a position measuring system (e.g. OPTOTRAK 3020, Northern Digital, Waterloo, Ontario, Canada). The central axis of the bended drill bit follows a real space trajectory, and the entry point is where this trajectory enters the target body. The entry point is fixed with respect to the target body, so once it has been digitized by a physical pointer (which may be the calibrated drill drive/drill bit combination itself), its position in space can be tracked. Furthermore, its position with respect to the drill drive can be computed. In conclusion, it is possible to track this one single point of the drill bit trajectory. The determination of the virtual space trajectory based on entry point tracking is called optical deflection sensing.

FlexDrill, together with the other instruments and the surgical object, is displayed in a 3D scene graph. Straight and deflected drill bit can be displayed simultaneously, or, alternatively, one of them alone. A guideline indicates the drill drive direction, simplifying correct handling. As an option, spherical tags can be set to mark trajectory points of interest such as drill bit tip or entry point. The deflected drill bit either can be displayed as a solid cylindrical structure of the drill bits dimensions, or as a line structure with an adjustable screen width.

FlexDrill compensates for the axial shift of the chuck jaws according to the drill bit diameter. Position, deflection, and other parameters such as the actual time can be logged to a file for later analysis of the drilling action.

The measurement of the position and orientation of the reference bases with respect to the three-dimensional on-site system of coordinates is performed with a position measurement device that is connected to the computer using software to evaluate the coordinates from the data received from the position measurement device. Position measurement devices comprising a computer are disclosed e.g. in the field of surgery in EP 0 359 773 SCHLÖNDORFF or U.S. Pat. No. 5,383,454 BUCHHOLZ.

The reference bases preferably comprise at least three markers that are non-collinearly arranged. The markers as well as the detectors of the position measurement device may be acoustic or electromagnetic effective means such as energy emitting, receiving or reflecting means. For instance as energy emitting means:

Light sources, particularly light emitting diodes (LEDs);

Infrared light emitting diodes (IRED's); or

Acoustic transmitters

or as energy receiving means:

Photodiodes; or

Microphones may be used. Other position measurement devices contain coils as energy emitting means and Hall-effect components as energy receiving means may be used as well.

A custom optoelectronic position measurement device may be used e.g. an OPTOTRAK 3020 System, Northern Digital, Waterloo, On., Canada. It preferably comprises:

an OPTOTRAK 3020 Position Sensor consisting of three one-dimensional charge-coupled devices (CCD) paired with three lens cells and mounted on a stabilised bar. Within each of the three lens cells, light from an infrared marker is directed onto a CCD and measured. All three measurements together determine—in real time—the three-dimensional location of the marker;

a system control unit;

a computer interface card and cables;

data collection and display software; and

a strober and marker kit.

The establishment of the mathematical description that specifies the spatial relationship between the tool holder and the body may be done by measuring the position and spatial orientation of a first reference base attached to the tool holder and of a second reference base attached to the body, whereby these measurements are performed with respect to an on-site three-dimensional system of coordinates. The deformation of the tool is preferably restricted to deformations due to a load application perpendicular to the central axis of the tool.

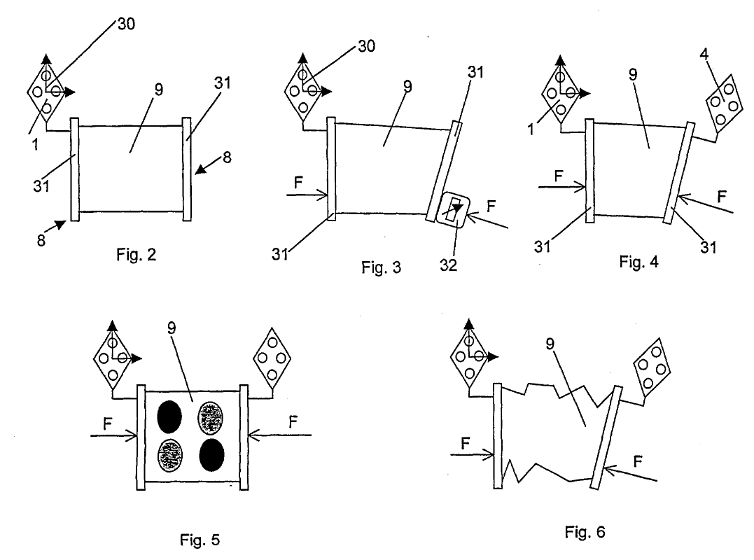

FIGS. 2-6 depict the application of the method according to the invention in case of forces and moments acting onto a cube 9. On two opposite faces, plates 31 made of a significantly stiffer material serve as tool holders 8 and are attached such that the plate deformation is negligible compared to the cube deformation. If no forces act on the cube 9, it can be represented by a rigid body, and every cube point is a fix point in the coordinate frame 30 of the dynamic reference base 1 attached to one of the plates 31 (FIG. 2). If moderate forces F are applied, the cube 9 deforms according to linear elastic theory. By a single dynamic reference base 1 only, the cube 9 cannot be tracked anymore because the cube points with respect to the coordinate frame 30 of the dynamic reference base 1 are not determined. But this determination can be accomplished by additional measurements. The forces F may be measured through a respective measurement instrument, e.g. a force gauge 32, and by applying linear elastic theory, the positions of the cube points can be calculated (FIG. 3). Alternatively, by a second dynamic reference base 4 attached at the second plate 31, the position of the second plate 31 can be tracked and again the positions of the cube points can be calculated (FIG. 4). Tracking of the object comprises the determination of the position of every point of the cube 9 in space and time. This may be done by a position measurement device 24 (FIG. 1) that is able to detect the position of the dynamic reference bases 1; 4 that are attached to the plates 31. A calibration procedure defines the object points with respect to the dynamic reference base 1. In order to track the cube 9 it is only necessary to know the object point positions in time and space with respect to the coordinate frame 30 connected to a dynamic reference base 1, but the points do not need to be fix points. Tracking the deforming cube 9 therefore requires a procedure that determines these cube point positions. Such a procedure measures kinematic parameters at the cube boundaries, and these boundary conditions in turn are used to determine the deformation of the cube 9 by the methods of continuum mechanics. If the forces F exceed a certain limit, linear elastic theory is not appropriate anymore, but the cube 9 might still deform in a predictable manner, such as buckling (FIG. 5). The theory to describe the cube deformation becomes much more complicated, but in principle it is still possible to track the cube 9. However, applying high forces F, the cube 9 sooner or later will deform in an unpredictable manner, and no tracking is possible anymore (FIG. 6).

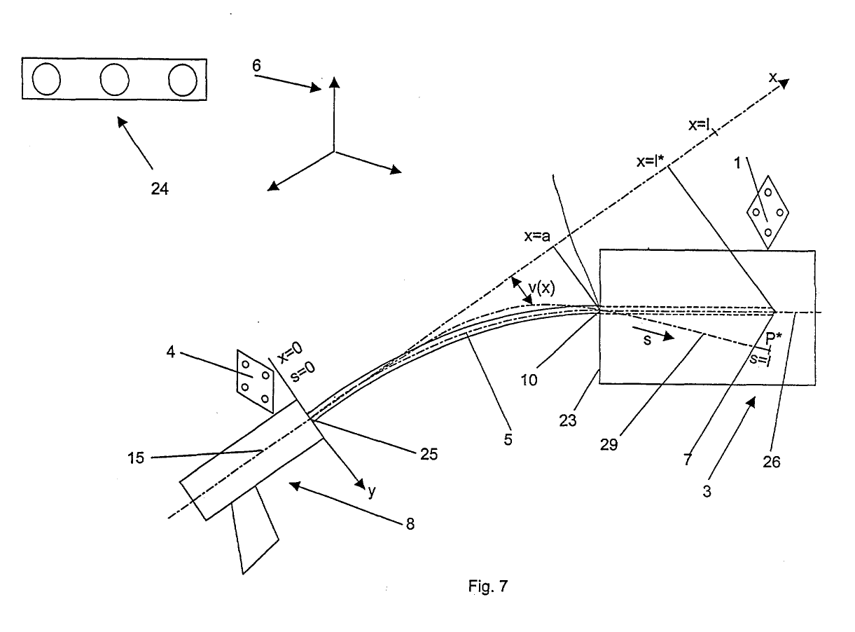

FIG. 7 represents the process of drilling a hole into a body 3 whereby the tool 5 is a drill bit that is deflected during the drilling process relative to the drill drive axis 15 (rigid body trajectory) through non-axial loads applied onto the tool holder 8. In this application of the method according to the invention the tool holder 8 is a drilling gear. At the tool holder 8 the first reference base 4 is situated and the body 3 is provided with the second reference base 1. The positions and spatial orientations of the reference bases 1;4 within a three-dimensional system of coordinates 6 are determined via a position measurement device 24. The central axis 26 of the tool 5 and the virtual space trajectory 29 may be represented at the display unit 22 of the computer 19 (FIG. 1). Thereby, the virtual space trajectory 29 is determined through the method according to the invention and approximates the central axis 26 of the deflected tool 5 also denoted as real space trajectory. The determination of the deflection of the tool 5 during the exemplary drilling process is effected as follows:

The drilling gear and the body 3 are preferably considered to be rigid bodies. As such, any point belonging to drilling gear and body 3 can be optically tracked as soon as its position is known with respect to the attached dynamic reference bases 1;4. The drill bit is assumed to bend according to linear elastic theory. The central axis 26 of the bended drill bit follows a real space trajectory and enters the body 3 at the entry point 10. The entry point 10 is fixed with respect to the body 3, so once it has been digitized e.g. with the free end 7 of the tool 5 (or any physical pointer), its position in space is given by tracking the dynamic reference base 1 at the body 3. By coordinate transformation to e.g. the on-site system of coordinates 6 the position of the entry point 10 can be determined with respect to the dynamic reference base 4 at the drilling gear. Therefore, it is possible to track the entry point 10 as single point of the central axis 26 of the drill bit. The determination of the virtual space trajectory 29 based on tracking the entry point 10 is called optical deflection sensing.

The entry point 10 can be tracked only after its position is known in the coordinate frame of the dynamic reference base 1 at the body 3. One possibility to digitize the entry point 10 is to use an arbitrary digitizing tool. The free end 7 of the drill bit subsequently may be positioned correctly onto the digitized point, e.g. using a tracked awl that marks the entry point 10 by a little hole. Otherwise, the drill bit itself may be used to digitize the entry point 10. The positioning of the free end 7 of the drill bit at the entry point 10 may be performed under direct visibility or by means of a computer assisted surgery system e.g. as disclosed in EP 0 359 773 SCHLÖNDORFF or U.S. Pat. No. 5,383,454 BUCHHOLZ.

The two steps to determine the virtual space trajectory 29 are first the determination of the boundary conditions for the drill bit at the fixed end 25 at the chuck of the drilling gear and at the entry point 10, and second the calculation of the beam deflection according to linear elastic theory. The entry point 10 divides the virtual space trajectory 29 in a free part with boundaries at the fixed end 25 at the chuck and at the entry point 10 and a target part where the virtual space trajectory 29 runs into the body 3. The boundary condition at the fixed end 25 is a zero deflection v(x=0)=0 and a fixed tangent direction of zero slope v′(x=0)=0. No loading acts on the free part of the drill bit. At the entry point 10, the boundary condition is a deflection v(x=a) according to the entry point tracking. The drill bit sticks in the hole drilled in the body 3, such that the body 3 can transmit forces and moments to the drill bit. Since the slope v′(x=a) is not known an assumption about the forces and moments at the entry point 10 has to be made, e.g. that only a shear force perpendicularly to the central axis 26 of the drill bit at the entry point 10 causes the drill bit bending. The target part of the drill bit is assumed to remain straight.

Once the boundary conditions are established, the virtual space trajectory 29 can be determined according to the FlexDrill concept. During drilling, the central axis 26 or real space trajectory of the drill bit varies with time, suggesting a dynamic character of drill bit kinematics. If mass inertia are neglected, the central axis 26 or real space trajectory reacts instantaneously on load changes, and the situation is a static one at every moment of time. For the static situation, linear elastic deformation of the drill bit leads to the Euler-Bernoulli theory of slender beams. If a cartesian coordinate frame xyz is defined such the origin is at the fixed end 25 of the drill bit at the chuck, the x-axis is the drill drive axis 15 (rigid body trajectory) and orientated against the body 3, and the xy-plane is given by the x-axis and the deflected free end 7 of the drill bit. In this coordinate frame, the coordinate vectors are [0,0,0]^{T} for the fixed end 25 of the drill bit at the chuck, [1,0,0]^{T} for the non-deflected f end 7 of the drill bit, [l,v(1),0]^{T} for the deflected free end 7 of the drill bit, and [a,v(a),0]^{T} the entry point 10. Thereby, a is the distance between the fixed end 25 of the drill bit and the entry point 10 and l is the overall length of the drill bit.

The x-axis coincides with the drill drive axis 15 and may also be denoted as a second virtual space trajectory which is computed according to the rigid body concept.

I* is the projection of the free end 7 of the drill bit onto the drill drive axis 15 (the position of the free end 7 of the drill bit on the virtual space trajectory 29 is computed according to the FlexDrill concept, i.e. the point P* is determined by numerical computation of the arclength s along the virtual space trajectory 29 and setting s=l).

The deflection v(x) of the virtual space trajectory 29 and the bending moment M_{z}(x) are linked by the differential equation v″(x)=M_{z}(x)/E1, where E is Young's modulus and l is the second moment of inertia of the drill bit cross section area. The coordinate frame xyz is not fix with respect to the drilling gear but rotates about the drilling gear axis according to the current direction of the drill bit deflection.

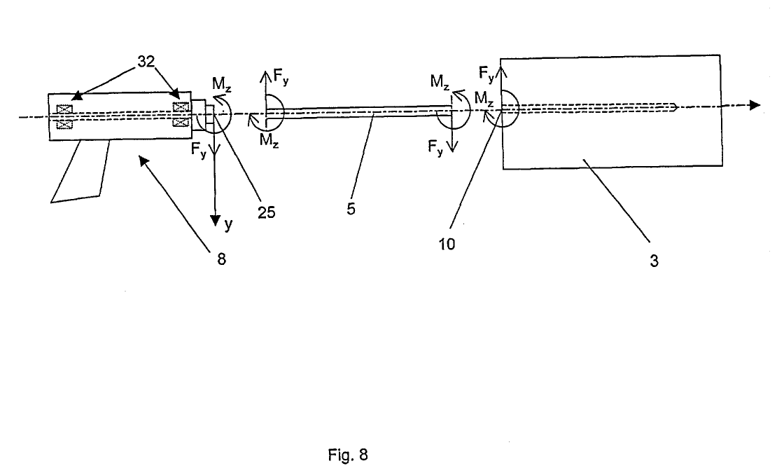

FIG. 8 represents an embodiment of the device according to the invention which differentiates from the embodiment shown in FIG. 7 that instead of an optical deflection sensing the determination of the deflection v(x) (FIG. 8) of the tool 5 is performed by means of force gauges 32 attached to the shaft or the housing of the tool holder 8 respectively the drilling gear. Via these force gauges 32 the load acting on the tool 5 at the entry point 10 can be determined. Once the load acting on the tool 5 at the entry point 10 is known the deflection v(x) (FIG. 8) may be calculated as follows:

It is supposed that the Euler-Bernoulli theory of slender beams is appropriate and that the components Fₓ and Mₓ can be neglected. F_{yz} and M_{yz} denote the projection of F and M, respectively, onto the yz-plane whereof the components Fₓ and M_{z} are shown in FIG. 9. In general, F_{yz} and M_{yz} will not be orthogonal. If indeed they are not, the trajectory does not lie in the xy-plane but intersects the xy-plane at the entry point, and the trajectory tangents at the fixed end 25 and at the entry point 10 are warped. The trajectory [x,v_{y}(x),vz(x)] can be decomposed in its projections [x,v_{y}(x),0] and [x,0,v_{z}(x)] onto the xy-plane and xz-plane, respectively, and the projections be analysed separately. For a single force,

v(x)=F(31x²−x³)/6E1

and for a single bending moment

v(x)=M x²/2E1

Therefore, it is

v_{y}(x)=[(31x²−x³)F_{y}+3M_{z}]/6E1

and

v_{z}(x)=[(31x²−x³)F_{z}+3M_{y}]/6E1

The formulas referring to the Euler-Bernoulli theory of slender beams as applied in the method according to the invention may also be looked up in: Dubbel Taschenbuch für den Maschinenbau 19. Auflage, Springer-Verlag PageS C19-C26.

While the present invention has been described with reference to one or more preferred embodiments, it should be kept in mind that variations from these are encompassed by the invention, whose scope is defined in the claims below.

What is claimed is:

1. A method of determining a deformation of an instrument comprising (i) a tool holder and (ii) a tool comprising a central axis, the tool having an un-deformed state, the method comprising the steps of:
(a) determining a position, in three-dimensional space, of a first point of a body to be operated on using the tool, wherein determining the position of the first point of the body comprises detecting energy radiated by each of at least three energy emitters, the energy passing between the at least three energy emitters and a detector, the at least three energy emitters and the detector being mechanically separate from one another;
(b) determining a position, in three-dimensional space, of a proximal portion of the tool, wherein the position of the proximal portion of the tool is determined based upon the position of the first point of the body;
(c) determining a position, in three-dimensional space, of a distal portion of the tool, wherein the step of (c) determining the position comprises detecting energy radiated by each of at least three energy emitters, the energy passing between the at least three energy emitters and a detector, the three energy emitters and the detector being mechanically separate from one another;
(d) determining a difference between (i) a relative position of the proximal portion of the tool with respect to the distal portion of the tool as determined from steps (b) and (c) and (ii) a relative position of the proximal portion of the tool with respect to the distal portion of the tool when the tool is in the un-deformed state.

2. The method of claim 1, wherein the step of (a) determining comprises contacting, with a proximal end of the tool, the first point of a body.

3. The method of claim 1, wherein the proximal portion of the tool is a point along the central axis of the tool.

4. The method of claim 1, wherein tool holder comprises a first set of three energy emitters and the energy detected in the step of (c) determining is from the first set of three energy emitters.

5. The method of claim 4, wherein the body comprises a second set of three energy emitters and the energy detected in the step of (a) determining is from the second set of energy emitters.

6. The method of claim 5, wherein the same detector is used to detect the energy in the step of (a) determining and the step of (c) determining.

7. The method of claim 1, wherein deformation of the tool results from a load application perpendicular to the central axis of the tool.

8. The method of claim 1, comprising displaying a representation of an un-deformed central axis of the tool and a deformed central axis of the tool.

9. The method of claim 1, wherein the tool is a drill bit.

10. A device for determining deformations of surgical instruments due to loads applied to the surgical instruments, comprising:
(a) a tool comprising a central axis, the tool having an un-deformed state;
(b) a tool holder connectible with the tool and with which tool holder the tool may be manipulated;
(c) a position measurement device configured to:
(i) determine a position, in three-dimensional space, of a first point of a body to be operated on using the tool, wherein the position measurement device comprises at least three energy emitters and, mechanically separated from the at least three energy emitters, a detector, wherein the position measurement device is configured to determine the position of the first point of the body based upon energy (1) radiated by each of at least three energy emitters and (2) detected by the detector;
(ii) determine, based upon the position of the first point of the body, a position, in three-dimensional space, of a proximal portion of the tool;
(iii) determine a position, in three-dimensional space, of a distal portion of the tool; and
(iv) determine a difference between (i) a relative position of the proximal portion of the tool with respect to the distal portion of the tool and (ii) a relative position of the proximal portion of the tool with respect to the distal portion of the tool in the un-deformed state.

11. The device of claim 10, comprising a reference base configured to be secured to the body, the reference base comprising the at least three energy emitters.

12. The device of claim 11, wherein the tool holder also comprises a marker comprising at least three energy emitters and wherein the processor is configured to determine the position of the distal portion of the tool based upon energy received from the at least three energy emitters of the marker of the tool holder.
